(19) United States
(12) Patent Application Publication
(10) Pub. No.: US 2023/0173198 A1
(43) Pub. Date: Jun. 8, 2023

(54) DRUG DELIVERY MEMBER INSERTION SENSING ASSEMBLIES, DRUG DELIVERY DEVICES, AND RELATED METHODS

(71) Applicant: AMGEN INC., Thousand Oaks, CA (US)

(72) Inventors: Mohammad Bonakdar, Waltham, MA (US); Daniel Eduardo Groszmann, Belmont, MA (US); Heejin Lee, Bedford, MA (US); Leonela Sabrina Vega Loaisa, Somerville, MA (US)

(21) Appl. No.: 17/911,545

(22) PCT Filed: Feb. 8, 2021
(86) PCT No.: PCT/US2021/017000

§ 371 Date: Sep. 14, 2022

Related U.S. Application Data
(60) Provisional application No. 62/990,133, filed on Mar. 16, 2020.

(51) Int. Cl. A61M 5/46; A61M 5/32

(52) U.S. Cl. CPC A61M 5/46; A61M 5/3293; A61M 5/322; A61M 2205/3306; A61M 2205/50

(57) ABSTRACT

Drug delivery member insertion depth sensing assemblies, drug delivery devices, and methods for determining an insertion depth of a drug delivery member are disclosed. The sensing assemblies can include electrical sensing assemblies with direct or indirect measurement components or optical sensing assemblies with one or more light sources and one or more photodiodes. A controller of the sensing assemblies can receive data associated with the drug delivery member in an insertion position. The data can then be correlated to an insertion depth of the drug delivery member.

### CROSS-REFERENCE TO RELATED APPLICATIONS

Priority is claimed to U.S. Provisional Patent Application No. 62/990,133, filed Mar. 16, 2020, the entire contents of which are hereby expressly incorporated herein by reference.

### FIELD OF THE DISCLOSURE

The present disclosure relates to drug delivery devices and, more particularly, drug delivery devices with electronic control.

### BACKGROUND

Drugs can be administered through the use of drug delivery devices such as autoinjectors or on-body injectors. Autoinjectors and on-body injectors may be used to help automate the injection and delivery or administration process, thereby simplifying the process for certain patient groups or sub-groups for which use of the syringe/vial combination or pre-filled syringe systems would be disadvantageous, whether because of physiological or psychological impediments.

Even after receiving specified training, however, some patients and/or caregivers can experience challenges while using autoinjectors and/or on-body injectors. Such challenges may relate to placement of the device on the person and/or device activation. The user may be uncertain if the device is located properly prior to operation. The user may inadvertently move the device before a full dose can be dispensed or may dispense the drug at a depth that is not optimal. The user may also be uncertain whether his/her sequence of actions has correctly operated the drug delivery device.

### SUMMARY

In accordance with a first aspect, a drug delivery device is disclosed that includes a housing, a primary container disposed within the housing, a drug delivery member fluidly coupled to the primary container, where the drug delivery member is movable between a retracted position disposed in the housing and an injection position at least partly extending out of the housing, and a wire having a first end and a second end, where the second end is electrically connected at a connection point adjacent to the drug delivery member and the connection point is fixed against movement relative to the housing. The drug delivery device further includes a controller in communication with the first end of the wire. The controller is configured to receive from the wire capacitance information associated with the drug delivery member in the injection position.

According to some forms, the controller can be configured to correlate the capacitance information to a depth that the drug delivery member has been inserted into a patient. In further forms, the drug delivery device can include a needle insertion mechanism. In these forms, the drug delivery member has an elongate configuration with a proximal end extending from the primary container and being fixed against movement with respect to the housing, an intermediate bend, and a distal end. The needle insertion mechanism is configured to move at least a portion of the distal end between the retracted position and the injection position and the second end of the wire is fixed to the connection point at the proximal end of the drug delivery member. In other further forms, the drug delivery device can include a pair of capacitor plates disposed within the housing and spaced from the drug delivery member, where the drug delivery member extends between the pair of capacitor plates. In these forms, the connection point is located on the pair of capacitor plates and the controller is in communication with the capacitor plates to receive capacitance information associated with the drug delivery member in the injection position. In further forms, the drug delivery device includes one or more dielectric members disposed between the capacitor plates and the drug delivery member, where the one or more dielectric members spaced outwardly from the drug delivery member.

In any of the above forms, the drug delivery member can include a cannula having a conductive portion and the connection point can be adjacent to the conductive portion of the cannula. Further, in some versions, the conductive portion of the cannula can be a conductive coating extending over at least a portion of an outer surface of the cannula.

In accordance with a second aspect, a method for determining an insertion depth of a drug delivery member is disclosed that includes moving a drug delivery member from a retracted position disposed in a housing of a drug delivery device to an injection position extending at least partly out of the housing, monitoring capacitance information associated with the drug delivery member in the injection position with a controller of the drug delivery device via a wire electrically connected at a connection point adjacent to the drug delivery member, where the connection point is fixed against movement relative to the housing, and correlating, with the controller, the capacitance information to a depth that the drug delivery member has been inserted into a patient.

According to some forms, the drug delivery member can have an elongate configuration with a proximal end fixed against movement with respect to the housing, an intermediate bend, and a distal end, and monitoring the capacitance information associated with the drug delivery member in the injection position with the controller can include monitoring capacitance information associated with the drug delivery member with the controller via a wire electrically connected at the connection point at the proximal end of the drug delivery member.

According to some forms, moving the drug delivery member from the retracted position disposed in the housing of the drug delivery device to the injection position extending at least partly out of the housing can include moving the drug delivery member between a pair of capacitor plates disposed within the housing and spaced from the drug delivery member, and monitoring the capacitance information for the drug delivery member in the injection position with the controller can include monitoring capacitance information for the drug delivery member as the drug delivery member is moved from the retracted position to the injection position with the controller via the wire electrically connected at the connection point adjacent the pair of capacitor plates. In further forms, the method can include spacing the capacitor plates from the drug delivery member with one or more dielectric members.

In any of the above forms, monitoring the capacitance information associated with the drug delivery member in the injection position with the controller of the drug delivery device can include monitoring capacitance information associated with a cannula with the controller via the wire electrically connected at the connection point adjacent to a conductive portion of the cannula.

In accordance with a third aspect, a drug delivery device is disclosed that include a housing, a hub movably disposed within the housing, a drug delivery member having a portion extending through and connected to the hub, a needle insertion mechanism operably coupled to the hub and configured to move the hub to drive the drug delivery member between a retracted position disposed in the housing and an injection position at least partly extending out of the housing, and a wire with a first end and a second end. At least a portion of the second end of the wire is fixed to the hub and electrically connected to the portion of the drug delivery member extending through the hub. The drug delivery device further includes a controller in communication with the wire to receive capacitance information associated with the drug delivery member in the injection position.

According to some forms, the controller can be configured to correlate the capacitance information to a depth that the drug delivery member has been inserted into a patient. In further forms, the drug delivery member can include a cannula having a conductive portion and the wire is electrically connected to the conductive portion of the cannula. If desired, the conductive portion of the cannula can be a conductive coating extending over at least a portion of an outer surface of the cannula.

In accordance with a fourth aspect, a method for determining an insertion depth of a drug delivery member is disclosed that includes moving a hub disposed within a housing of a drug delivery device with a needle insertion mechanism to thereby drive a drug delivery member from a retracted position disposed in a housing of a drug delivery device to an injection position extending at least partly out of the housing, the drug delivery member having a portion extending through and connected to the hub, monitoring capacitance information associated with the drug delivery member in the injection position with a controller of the drug delivery device via a wire having a first end and a second end, where at least a portion of the second end is fixed to the hub and electrically connected to the portion of the drug delivery member extending through the hub, and correlating, optionally with the controller, the capacitance information to a depth that the drug delivery member has been inserted into a patient.

According to some forms, monitoring the capacitance information associated with the drug delivery member in the injection position with the controller of the drug delivery device can include monitoring capacitance information associated with a cannula with the controller via a wire fixed to the hub and electrically connected to a conductive portion of the cannula.

In accordance with a fifth aspect, a drug delivery device is disclosed that includes a hub and a drug delivery member fixed to the hub and having a proximal opening and a distal opening. The proximal opening is disposed within the hub and in communication with the distal opening. The drug delivery device further includes a light source oriented to project light into the proximal opening of the drug delivery member, out of the distal opening of the drug delivery member, and into the tissue of a patient with the drug delivery member in an injection position, and a photodiode is oriented to receive light radiating through the tissue of the patient adjacent to the drug delivery member when the drug delivery member is in the injection position. A controller is in communication with the photodiode to receive data associated with the light received.

According to some forms, the controller can be configured to correlate the data to a depth that the drug delivery member has been inserted into the tissue of the patient. In further forms, the drug delivery device can include a housing, a primary container disposed within the housing, and a flowpath fluidly coupling the primary container to the drug delivery member. In a first version, the drug delivery member extends through the hub and includes a bend disposed within the hub with the proximal opening extending therethrough and a distal end extending from the bend through a bottom surface of the hub. In a second version, the drug delivery device includes an inlet conduit mounted to hub, where the hub includes an internal cavity, the inlet conduit fluidly couples the flowpath to the internal cavity of the hub, and the drug delivery member extends from the internal cavity through a bottom surface of the hub, such that the proximal opening of the drug delivery member fluidly couples the drug delivery member to the internal cavity. In either version, the photodiode can be mounted to an upper surface of the hub and/or can be mounted to a bottom wall of the housing adjacent to a drug delivery member opening extending therethrough. In other further forms, the drug delivery device can include a primary container. In these forms, the drug delivery member can be a needle, the hub can be fixedly mounted to a distal end of the primary container, the light source can be an array of light sources carried by the hub, and the photodiode can be an array of photodiodes extending around and adjacent to the distal end of the primary container. The array of light sources and the array of photodiodes are non-aligned to provide generally unobstructed paths toward a distal end of the needle.

In accordance with a sixth aspect, a method for determining an insertion depth of a drug delivery member of a drug delivery device is disclosed that includes inserting a drug delivery member into the tissue of a patient, where the drug delivery member has a proximal end fixed to a hub and a distal end opposite the proximal end, and emitting light from a light source carried by the drug delivery device into a proximal opening at the proximal end of the drug delivery member, out of a distal opening at the distal end of the drug delivery member, and into the tissue of the patient. The method further includes receiving, with a photodiode, light radiating through the tissue of the patient adjacent to the drug delivery member, receiving, at a controller, data regarding the light received by the photodiode, and correlating, optionally with the controller, the data received at the controller to a depth that the drug delivery member has been inserted into the tissue of the patient.

In a first version, inserting the drug delivery member fixed to the hub of the drug delivery device into the tissue of the patient can include moving the hub having the drug delivery member extending therethrough with a needle insertion mechanism. In a second version, inserting the drug delivery member fixed to the hub of the drug delivery device into the tissue of the patient can include moving a hub having an internal cavity and the drug delivery member extending from the internal cavity through a bottom surface of the hub with a needle insertion mechanism, where the proximal opening of the drug delivery member fluidly couples the drug delivery member to the internal cavity. In either version, receiving the light through the tissue of the patient with the photodiode can include receiving light through the tissue of the patient with a photodiode mounted to an upper surface of the hub or receiving light through the tissue of the patient with a photodiode mounted to a bottom wall of a housing of the drug delivery device.

According to some forms, inserting the drug delivery member fixed to the hub of the drug delivery device into the tissue of the patient can include inserting a needle fixed to the hub fixedly mounted to a distal end of a primary container into the tissue of a patient, emitting light from the light source can include emitting light from an array of light sources carried by the hub, and receiving the light through the tissue of the patient with the photodiode can include receiving light through the tissue of the patient with an array of photodiodes extending around and adjacent to the distal end of the primary container, where the array of light sources and the array of photodiodes are non-aligned to provide generally unobstructed paths toward a distal end of the needle.

### BRIEF DESCRIPTION OF THE DRAWINGS

FIG. 1 is a diagrammatic view of an autoinjector drug delivery device according to an embodiment of the present disclosure.

FIG. 2 is a diagrammatic view of an on-body drug delivery device according to an embodiment of the present disclosure.

FIG. 3 is a sectional view of a first example electrical sensing assembly for a drug delivery device according to an embodiment of the present disclosure.

FIG. 4 is a sectional view of a second example electrical sensing assembly for a drug delivery device according to an embodiment of the present disclosure.

FIG. 5 is a sectional view of a third example electrical sensing assembly for a drug delivery device according to an embodiment of the present disclosure.

FIG. 6 is a sectional view of a first example optical sensing assembly for a drug delivery device according to an embodiment of the present disclosure.

FIG. 7 is a sectional view of a second example optical sensing assembly for a drug delivery device according to an embodiment of the present disclosure.

FIG. 8 is a sectional view of a third example optical sensing assembly for a drug delivery device according to an embodiment of the present disclosure.

### DETAILED DESCRIPTION

Concepts for sensing systems have been proposed for measuring the insertion depth of a drug delivery member, such as a needle or soft cannula, of a drug delivery device into the skin of a patient by taking measurements and running algorithms to process the data. The coupling between sensing systems and a drug delivery member of the drug delivery device, however, is challenging. As such, example configurations for the coupling and/or integration of sensing systems into or with drug delivery members of a drug delivery device, such as autoinjectors or on-body injectors, are provided herein.

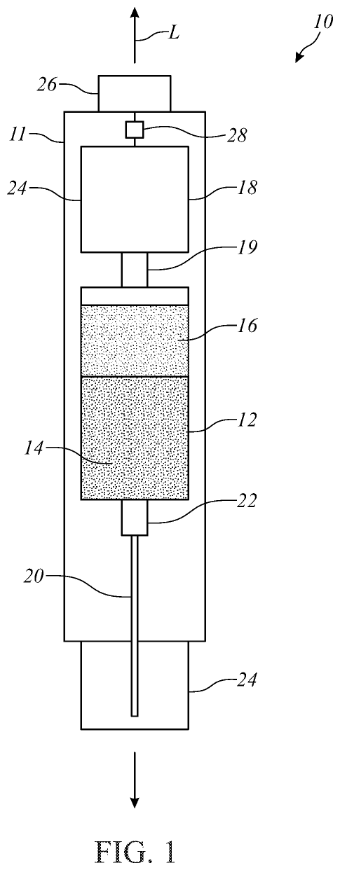

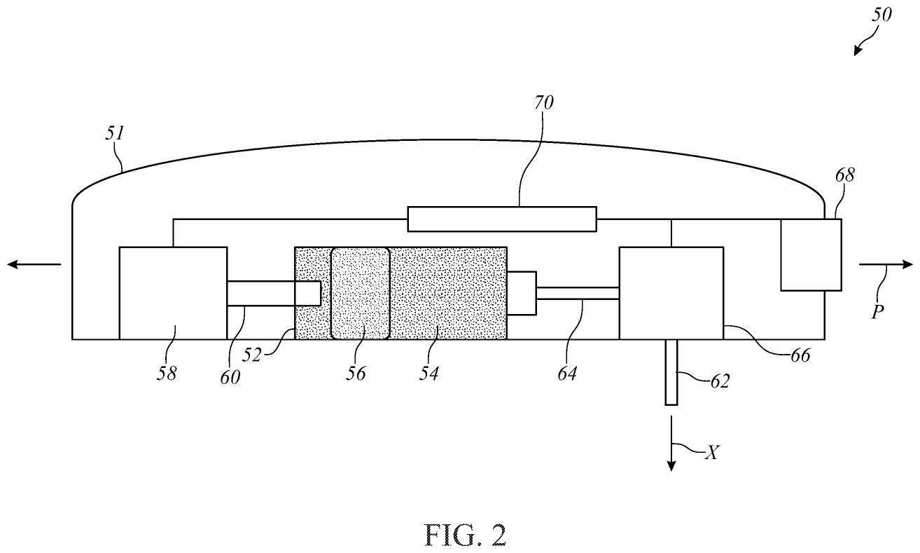

Before providing details of example sensing systems, example drug delivery devices are shown in FIGS. 1 and 2. In some versions as illustrated in FIG. 1, drug delivery devices 10, such as autoinjectors, can have a vertically oriented configuration with some or all drug delivery components, including an injection assembly, disposed in stacked relation along a longitudinal axis L within a housing 11 of the devices 10. As a more specific example, the devices 10 can be configured to operate and inject a user with the device 10 oriented generally perpendicular to a skin surface of the user. The drug delivery components can include a primary container 12, such as a reservoir, having a drug 14 contained therein, a stopper 16 disposed within the primary container 12 and slidably movable therein along the longitudinal axis L, a needle 20 having a distal end oriented along the longitudinal axis L, and a flow path 22 fluidly coupling the primary container 12 to the needle 20. The components can further include an injection assembly that includes a drive mechanism 18 coupled to a plunger 19 to drive the stopper 16 through the primary container 12 and a needle insertion mechanism (NIM) 24 configured to insert the needle 20 to a desired subcutaneous depth within the user. By some approaches, the NIM 24 can be a retractable needle guard to expose the needle 20 or a drive mechanism to longitudinally move the needle a desired distance. For example, the drive mechanism 18 can be configured to drive both movement of the stopper 16 and the needle 20 by moving some or all of the primary container 12, flow path 22, and needle 20. As commonly configured, one or more of the components of the device 10, such as the drive mechanism 18 and NIM 24, can be operable in response to actuation of a user input device 26 accessible on an exterior of the housing 11. Suitable drive mechanisms include, but are not limited to, springs, gas sources, phase changing materials, motors, or other electromechanical systems. Pursuant to this, the device 10 can include electronic components, such as a controller 28, to control operation of one or more of the drug delivery components. It will be understood that although FIG. 1 shows the components centered along the longitudinal axis L, one or more of the components can be disposed off-center from the longitudinal axis L within the housing 11 and still be considered to be in a stacked relation. In one example, an autoinjector drug delivery device having drug delivery components in a stacked relation corresponds to the primary container 12 co-axially aligned with the needle 20. In some versions, the device 10 can include a cap assembly that includes a cap housing and a remover. The device 10 can further include a needle shield disposed over at least a portion of a distal end of the needle 20 in a storage state, where the needle shield is engaged and retained by the remover. The needle shield can then be removable by extraction of the cap assembly from the device 10. Example autoinjector devices are described in U.S. Ser. No. 62/447,174, filed Jan. 17, 2017, which is hereby incorporated by reference herein.

In other versions as illustrated in FIG. 2, drug delivery devices 50, such as on body injectors, can have a horizontally oriented configuration with drug delivery components disposed generally along a horizontal plane P within a housing 51 of the devices 50. With these devices 50, the housing 51 has a low profile with a larger width than height so that when a user positions the housing 51 on the skin, the components are spread out over an area of the skin rather than stacked as with the above embodiments. The drug delivery components can include a primary container 52, such as a reservoir, having a drug 54 contained therein, which can be removably disposed within the housing 51, a stopper 56 disposed within the primary container 52 and slidably movable therein along the horizontal plane P, a drive mechanism 58 coupled to a plunger 60 to drive the stopper 56 through the primary container 52, a needle and/or soft cannula 62 oriented along an axis X that extends generally transverse, such as perpendicular or at an angle with respect thereto, to the horizontal plane P, a flow path 64 fluidly coupling the primary container 52 to the needle 62, and a NIM 66 configured to insert the needle 62 to a desired subcutaneous depth within the user. As commonly configured, one or more of the components of the device 50, such as the drive mechanism 58 and NIM 66, can be operable in response to actuation of a user input device 68 accessible on an exterior of the housing 51. Pursuant to this, the device 50 can include electronic components, such as a controller 70, to control operation of one or more of the drug delivery components. As described above, the device 50 of this form can also include a cap assembly that includes a cap housing and a remover. The device 50 can further include a needle shield disposed at least over a portion of a distal end of the needle 62 in a storage state, where the needle shield is engaged and retained by the remover. The needle shield can be removable by extraction of the cap assembly from the device 50. Of course, it will be understood that some components can be disposed partially or entirely above or below the horizontal plane P extending generally centrally through the housing 51 and still be considered to have a horizontally oriented configuration. Suitable drive mechanisms include, but are not limited to, springs, gas sources, phase changing materials, motors, or other electromechanical systems. Example on body injector devices are described in U.S. Ser. No. 62/536,911, filed Jul. 25, 2017, which is hereby incorporated by reference herein.

Sensing systems for drug delivery devices 10, 50 can measure the insertion depth of a drug delivery member 20, 52 either electrically or optically. In one form, electrical sensing systems 100 measure the insertion depth by measuring the capacitance between the inserted drug delivery member 20, 52 and an electrode 102 in contact with the skin 104 of the patient. Using algorithms, the sensing system 100, either locally or remotely, correlates the measured mutual capacitance to the inserted depth of the drug delivery member 20, 52. Alternatively, an electrical sensing system 100 can measure the impedance (self-capacitance) as a function of drug delivery member 20, 52 depth without using an electrode against the skin of a patient. In another form, optical sensing systems 200 measure the insertion depth of a drug delivery member 20, 52 by shining light with a light source 202 through the penetrating drug delivery member 20, 52 into the tissue of the patient and receiving back scattered light radiating through the tissue of a patient with one or more photodiodes 206 on or adjacent to the skin 204 of the patient. For example, the photodiodes 206 measure an intensity of the light and transmit data associated with the intensity of the light. Using algorithms, the sensing system 200, either locally or remotely, correlates the insertion depth of the drug delivery member 20, 52 to the intensity of the received back scattered light.

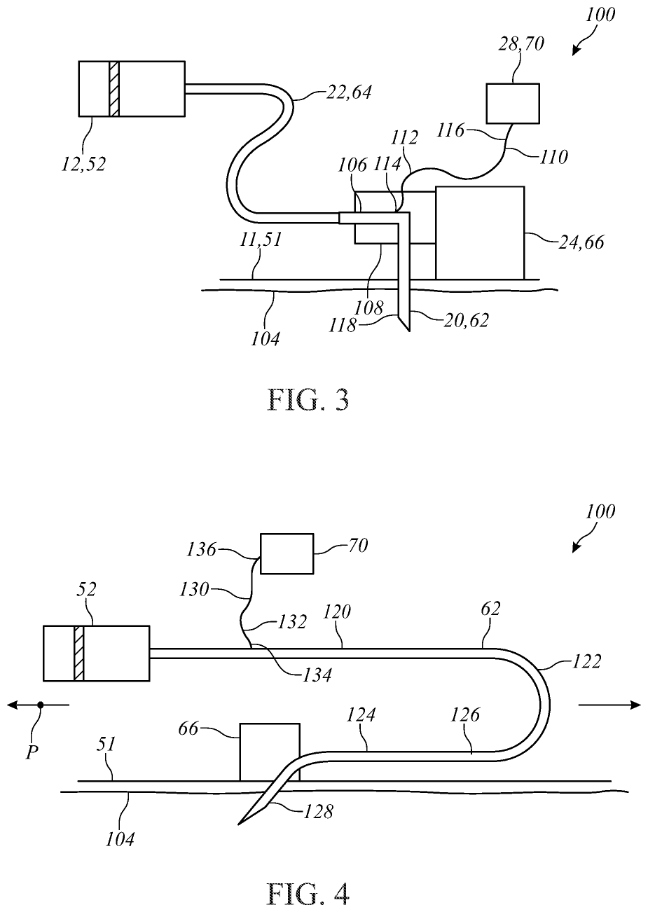

A first example electrical sensing assembly 100 for a drug delivery device 10, 50 is shown in FIG. 3. In this form, the drug delivery member 20, 62 is a needle fluidly coupled to the primary container 12, 52 by the flowpath 22, 64, which can be flexible tubing as shown, or rigid. Further, the needle 20, 62 includes a portion 106 extending through and connected to a hub 108 received within the device 10, 50. The hub 108 is manipulated by the NIM 24, 66 to move the needle 20, 62 from a retracted, storage position disposed in the housing 11, 51, to an injection position at least partly extending out of the housing 11, 51. The needle 20, 62 can have a bent configuration, as shown in FIG. 3 with the needle 20, 62 entering the hub 108 from a side and exiting the hub 108 through a bottom or can have a straight configuration with the needle 20, 62 entering the hub 108 from the top and exiting through the bottom. It will be understood that either configuration can be utilized in an autoinjector 10 or an on-body injector 50.

Advantageously, the portion 106 of the needle 20, 62 disposed within the hub 108 is fixed relative to the hub 108. With this configuration, a wire 110 can have a first end 112 directly electrically connected to portion 106 of the needle 20, 62 extending through the hub 108 and fixed to the hub 108 at a fixed point 114, such that movement of the hub 108 and needle 20, 62 during an injection operation will not loosen the connection. In one version, the first end 112 of the wire 110 can be directly fixed to the needle 20, 62. A second end 116 of the wire 110 can be in communication with the controller 28, 70, which can analyze the capacitance information associated with the needle 20, 62 in the injection position provided by the wire 110 and correlate the capacitance information to a depth that the needle 20, 62 has penetrated the skin 104 of the patient. Alternatively or additionally, the capacitance information can be sent to a remote controller for analysis and correlation. The wire 110 can be flexible to accommodate the movement of the hub 108 and needle 20, 62 during operation of the device 10, 50 relative to the controller 28, 70. The direct electrical connection between the needle 20, 62 and the controller 28, 70 provides a reliable source of capacitance information without the risk of the connection of the wire 110 to the needle 20, 62 failing or providing intermittent results. In one version, the needle 20, 62 can be made from stainless steel.

A second example electrical sensing assembly 100 for a drug delivery device 50 is shown in FIG. 4. In this form, the needle 62 is directly fluidly connected to the primary container 52. As such, the needle 62 provides the flowpath 64. The needle 62 of this form has an extended, elongate configuration with a fixed, proximal end 120 extending from the primary container 52, an intermediate bend 122, and a movable, distal end 124. For example, the proximal end 120 can extend generally parallel, e.g., within 5 degrees or within 10 degrees, to the horizontal plane P and the bend 122 can be a 180 degree bend, such that at least a portion 126 of the distal end 124 extends backward generally parallel to the horizontal plane P. The distal end 124 can also include an angled portion 128 configured to be moved to an exterior of the housing 51 during an injection operation. The angled portion 128 can extend at an angle generally transverse to the plane P, such as between 30 degrees and 45 degrees, between 45 degrees and 60 degrees, between 60 degrees and 90 degrees. In this form, the NIM 66 can manipulate the distal end 124 of the needle 62 to move the angled portion 128 from a retracted, storage position disposed in the housing 51, to an injection position at least partly extending out of the housing 51. The bend 122 allows the distal end 124 to be moved relative to the fixed, proximal end 120.

Advantageously, a wire 130 can have a first end 132 and a second end 136. The second end 136 can be electrically connected to the needle 62 at a connection point 134 adjacent to the proximal end 120 of the needle 62. Advantageously, the connection point 134 is fixed against movement relative to the housing 51 due to the fixed configuration of the proximal end 120 of the needle 62. In one version, the second end 136 can be directly fixed to the needle 62, such that movement of the distal end 124 of the needle 62 during an injection operation will not loosen the connection. The first end 136 of the wire 130 can be in communication with the controller 70, which can analyze the capacitance information provided by the wire 130 associated with the needle 62 in the injection position and correlate the capacitance information to a depth that the needle 62 has penetrated the skin 104 of the patient. Alternatively or additionally, the capacitance information can be sent to a remote controller for analysis and correlation. Given that the proximal end 120 of the needle 62 and controller 70 are fixed with respect to one another and, the wire 130 can have a rigid or fixed configuration within the housing 51. Of course, the wire 130 can have a flexible configuration if desired. The direct electrical connection between the needle 62 and the controller 70 provides a reliable source of capacitance information without the risk of the connection of the wire 130 to the needle 62 failing or providing intermittent results. In one version, the needle 62 can be made from stainless steel.

A third example electrical sensing assembly 100 for a drug delivery device 10, 50 is shown in FIG. 5. Although this form is shown in FIG. 5 with reference to an autoinjector 10, it will be understood that the assembly 100 can readily be incorporated into an on-body injector 50. In this form, the NIM 24, 66 moves the needle 20, 62 between a retracted, storage position disposed in the housing 11, 51 and an injection position with a distal end of the needle 20, 62 at least partly extending out of the housing 11, 51. The electrical sensing assembly 100 of this form provides contactless monitoring of the needle 20, 62. As shown, the sensing assembly 100 includes a pair of capacitor plates 140 spaced apart from one another with the needle 20, 62 extending therebetween. If desired, the assembly 100 can further include one or more dielectric members 142 that are disposed between the capacitor plates 140 and the needle 20, 62. The dielectric members 142 are spaced from the needle 20, 62 to allow the needle 20, 62 to freely move during an injection operation, while also spacing the capacitor plates 140 from the needle 20, 62 a fixed distance to ensure a consistent reading during operation.

As shown, the assembly 100 can further include one or more wires 144 electrically coupled to the capacitor plates 140 and in communication with the controller 28, 70, which can analyze the capacitance information associated with the needle 20, 62 in the injection position provided by the wire 144 and capacitors 140. For example, the wire 144 can have a first end in communication with the controller 28, 70 and a second end electrically connected at a connection point adjacent to the needle 20, 62, i.e., a connection point to the capacitor plates 140. Advantageously, the connection point is fixed against movement relative to the housing 11, 51 due to the fixed configuration of the capacitor plates 140. The controller 28, 70, or optionally a remote controller, can then correlate the capacitance information to a depth that the needle 20, 62 has penetrated the skin 104 of the patient. Given that the capacitor plates 140 and controller 70 are fixed with respect to one another, the wiring 144 can have a rigid or fixed configuration within the housing 11, 51. Of course, the wiring 144 can have a flexible configuration if desired. The contactless monitoring of the needle 20, 62 provided by the capacitor plates 140 allows needle movement without compromising the electrical connection of the sensing assembly 100. By being mounted a fixed distance from the needle 20, 62, the capacitor plates 140 provide a reliable source of capacitance information without interfering with the operation and movement of the needle 20, 62. The sensing assembly 100 of this form is particularly advantageous for autoinjectors 10 or on-body injectors 50 having a short needle length, while also requiring needle movement for an injection operation. In one version, the needle 62 can be made from stainless steel.

In any of the above forms, the drug delivery member 20, 62 can include or be a cannula, such as a soft cannula. The cannula 20, 62 can be made conductive by coating an outer surface 150 thereof with a conductive material. For example, the conductive material can be gold or platinum. The conductive material can be coated on the outer surface 150 of the cannula 20, 62 by any suitable method, such as physical vapor deposition (PVD) or atomic layer deposition (ALD). In another version, the cannula 20, 62 could be made from polymer nanocomposites doped with carbon nanotube or metallic nanoparticles to make the cannula 20, 62 electrically conductive. With either of these configurations, the capacitor plates 140 can monitor the cannula 20, 62 as it is moved therebetween, as described above.

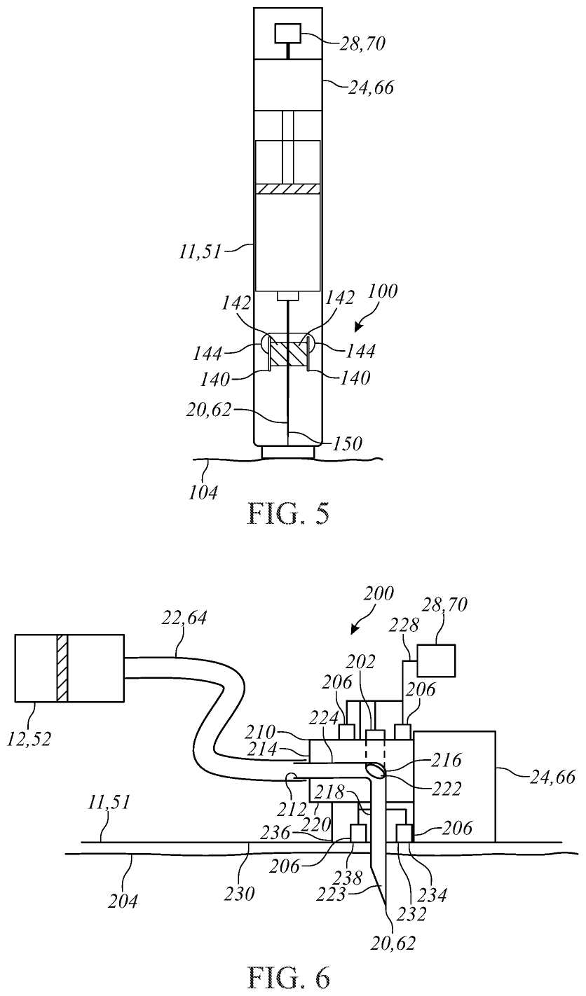

A first example optical sensing assembly 200 for a drug delivery device 10, 50 is shown in FIG. 6. In this form, the drug delivery member 20, 62 is a needle fluidly coupled to the primary container 12, 52 by the flowpath 22, 64, which can be flexible tubing as shown. Further, the needle 20, 62 extends through and is fixed to a hub 210 received within the device 10, 50 and the hub 210 is configured to be manipulated by the NIM 24, 66 to move the needle 20, 62 from a retracted, storage position disposed in the housing 11, 51, to an injection position at least partly extending out of the housing 11, 51. As shown, the needle 20, 62 includes a proximal end 212 that enters the hub 210 through a side 214, a bend 216 disposed within the hub 210, and a distal end 218 that exits the hub 210 through a bottom 220. In one example, the bend 216 of the needle 20, 62 can be a generally, e.g., within 5 degrees or within 10 degrees, 90 degree bend. In one version, the needle 20, 62 can be stainless steel.

As shown, the sensing assembly 200 further includes the light source 202 that is oriented and configured to project light into needle 20, 62 through a proximal opening 222 of the needle 20, 62 and through a distal opening 223 in communication with the proximal opening 222 to project light into the tissue of a patient after the distal end 218 of the needle 20, 62 has been inserted therein. In this form, the proximal opening 222 is provided through an upper surface 224 of the bend 212 that is aligned over the distal end 218 thereof. Further, the hub 210 can be configured to provide light access to the opening 222, such that a light can be projected through the hub 210 and opening 222, and into the distal end 218 of the needle 20, 62. For example, the hub 210 can be made from a transparent material, e.g., plastic, or an opaque material with a bore aligned with the needle opening 222 while maintaining the fluid tight nature of the needle 20, 62 with a transparent covering or shield. In one version, the light source 202 can be mounted to the hub 210 to move along therewith. In such a configuration, a wire 228 electrically connected to the light source 202 can be flexible to allow the light source 202 to move freely with the hub 210. In another version, the light source 202 can be fixedly mounted within the housing 11, 51 and the wire 228 can be fixed/rigid or flexible, as desired. In another form, the drug delivery member 20, 62 can include a soft cannula with a needle acting as a trocar. With this configuration, the light projected by the light source 202 can pass through the needle 20, 62 to the cannula. It will be understood that the above configurations can be utilized in an autoinjector 10 or an on-body injector 50.

In some versions, the assembly 200 can further include light focusing components to direct light into the needle 20, 62 and avoid scattering. For example, one or more lenses can be disposed within a path of light projected from the light source 202, such as mounted to or within the hub 210 and/or to or within the housing 11, 51, a reflective material can be disposed forwardly of the light source 202, such as within the bore of the hub 210, or the like.

As discussed above, the optical sensing assembly 200 receives back scattered light radiating through the tissue of a patient adjacent to the needle 20, 62 in an injection position by means of one or more photodiodes 206 on or adjacent to the skin 204 of the patient. The photodiodes 206 are electrically coupled to and in communication with the controller 28, 70 to provide data associated with the light received. The controller 28, 70, or optionally a remote controller, can then correlate the data to a depth that the needle 20, 62 has penetrated the skin 204 of the patient. In a first form, the photodiodes 206 can be disposed adjacent to the light source 202 with the light source 202 in a generally central location with respect thereto. The photodiodes 206 can either be coupled to the hub 210 to move along therewith or be mounted in fixed position within the housing 11, 51. With this configuration, the photodiodes 206 detect back scattered light through the hub 210, which can be made from transparent material or have one or more bores extending therethrough aligned with the photodiodes 206 as discussed above. In a second form, the photodiodes 206 can be coupled to a bottom wall 230 of the housing 11, 51 adjacent to a drug delivery member opening 232 extending therethough. For example, the bottom wall 230 can include a transparent portion 234 extending around the opening 232 and the photodiodes 206 can be mounted within the housing 11, 51 external, optically-transparent casing 236 with the hub 210 received therein, or the photodiodes 206 can be mounted within openings 238 of the bottom wall 230 to be directly adjacent the skin 204 of the patient.

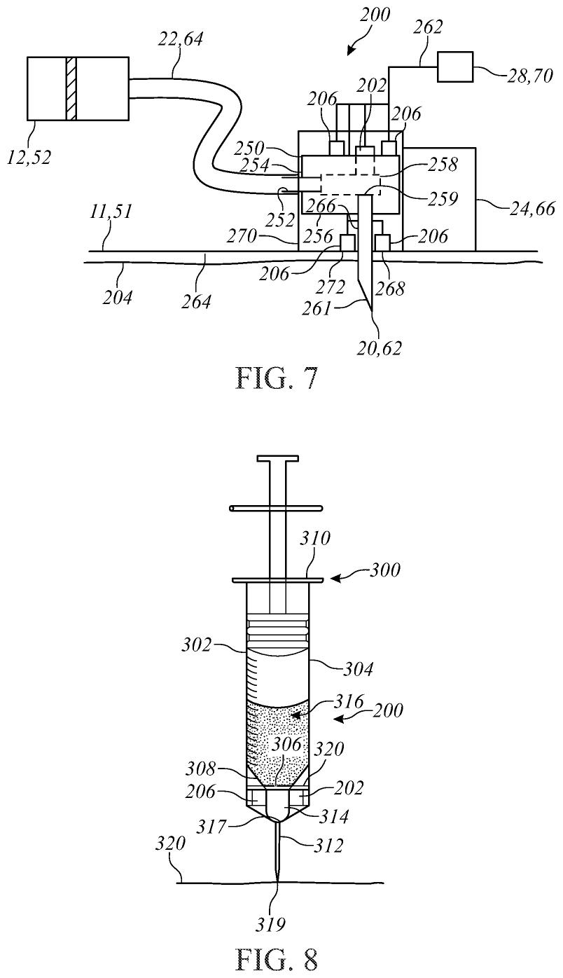

A second example optical sensing assembly 200 for a drug delivery device 10, 50 is shown in FIG. 7. In this form, the drug delivery member 20, 62 is a needle fluidly coupled to the primary container 12, 52 by the flowpath 22, 64, which can be flexible tubing as shown or rigid. Rather than a needle extending through a hub 250, as with the above form, in the assembly 200 of this form includes a separate inlet conduit 252 that enters the hub 250 through a side 254 and the needle 20, 62 exits the hub 250 through a bottom 256. Further, the hub 250 defines an internal cavity 258 that fluidly connects the inlet conduit 252 to the needle 20, 62. As with the above form, the hub 250 is configured to be manipulated by the NIM 24, 66 to move the needle 20, 62 from a retracted, storage position in the housing 11, 51, to an injection position at least partly extending out of the housing 11, 51. In one version, the needle 20, 62 can be stainless steel.

As shown, the sensing assembly 200 further includes the light source 202 that is oriented and configured to project light into the needle 20, 62 through a proximal opening 259 of the needle 20, 62 and through a distal opening 261 in communication with the proximal opening 259 to project light into the tissue of a patient after the needle 20, 62 has been inserted therein. In this form, the needle 20, 62 extends along a longitudinal axis and the hub 250 can be configured to provide light access thereto, such that a light can be projected through the hub 250 and into the needle 20, 62. For example, the hub 210 can be made from a transparent material, e.g., plastic, or an opaque material with a bore aligned with the needle opening 259 while maintaining the fluid tight nature of the needle 20, 62 with a transparent covering or shield. As shown, the light source 202 is aligned with the proximal opening 259 of the needle 20, 62, such that light projected by the light source 202 enters the needle 20, 62 and transfers to the tissue of the patient after the needle 20, 62 has been inserted therein. In one version, the light source 202 can be mounted to the hub 250 to move along therewith. In such a configuration, a wire 262 electrically connected to the light source 202 can be flexible to allow the light source 202 to move freely with the hub 250. In another version, the light source 202 can be fixedly mounted within the housing 11, 51 and the wiring 262 can be fixed/rigid or flexible, as desired. In another form, the drug delivery member 20, 62 can include a soft cannula with a needle acting as a trocar. With this configuration, the light projected by the light source 202 can pass through the needle 20, 62 to the cannula. It will be understood that the above configurations can be utilized in an autoinjector 10 or an on-body injector 50.

In some versions, the assembly 200 can further include light focusing components to direct light into the needle 20, 62 and avoid scattering. For example, one or more lenses can be disposed within a path of light projected from the light source 202, such as mounted to or within the hub 250 and/or to or within the housing 11, 51, a reflective material can be disposed forwardly of the light source 202, such as within the bore of the hub 250, or the like.

As discussed above, the optical sensing assembly 200 receives or measures back scattered light radiating through the tissue of a patient when the needle 20, 62 is in the injection position by means of one or more photodiodes 206 on or adjacent to the skin 204 of the patient. The photodiodes 206 are electrically coupled to and in communication with the controller 28, 70 to provide data associated with light received thereto. The controller 28, 70, or optionally a remote controller, can then correlate the data to a depth that the needle 20, 62 has penetrated the skin 204 of the patient. In a first form, the photodiodes 206 can be disposed adjacent to the light source 202 with the light source 202 in a generally central location with respect thereto. The photodiodes 206 can either be coupled to the hub 250 to move along therewith or be mounted in fixed position within the housing 11, 51. With this configuration, the photodiodes 206 detect back scattered light through the hub 250, which can be made from transparent material or have one or more bores extending therethrough aligned with the photodiodes 206 as discussed above. In a second form, the photodiodes 206 can be coupled to a bottom wall 264 of the housing 11, 51 adjacent to a drug delivery member opening 266 extending therethough. For example, the bottom wall 264 can include a transparent portion 268 extending around the opening 266 and the photodiodes 206 can be mounted within the housing 11, 51, such as embedded within an external, optically-transparent casing 270 with the hub 250 received therein, or the photodiodes 206 can be mounted within openings 272 of the bottom wall 230 to be directly adjacent the skin 204 of the patient.

A third example optical sensing assembly 200 for a pre-filled syringe 300 is shown in FIG. 8. The syringe 300 includes a primary container in the form of a barrel or reservoir 302 containing a fluid therapeutic product. The barrel 302 has an annular sidewall 304 extending between a dispensing opening 306 at a distal end 308 and an open proximal end 310. At the distal end 308 of the barrel 302, the syringe 300 includes a needle 312 fixed to a hub 314 coupled to the barrel 302, such that the needle 312 is fluidly coupled with an interior 316 of the barrel 302.

As shown, the sensing assembly 200 further includes an array of light sources 202 that are oriented and configured to project light into the needle 312 through a proximal opening 317 of the needle 20, 62 disposed within the hub 314 and through a distal opening 319 in communication with the proximal opening 317 to project light into the tissue of a patient after the needle 312 has been inserted therein. In this form, the needle 312 extends along a longitudinal axis and the hub 314 and/or barrel distal end 308 can be configured to provide light access thereto, such that a light can be projected through the hub 314 and/or barrel distal end 308 and into the needle 312. For example, the hub 314 and/or barrel distal end 308 can be made from a transparent material, e.g., plastic, or an opaque material with a bore aligned with the needle opening 317 while maintaining the fluid tight nature of the needle 312 and barrel interior 316 with a transparent covering or shield. The array of light sources 202 can extend around or within the hub 314 or barrel distal end 308.

The photodiodes 206 of the assembly 200 can be disposed in an array extending around or within the needle hub 314 or barrel distal end 308. The light sources 202 and photodiodes 206 can be positioned so that individual photodiodes 206 do not block light projected from the light sources 202 and so that individual light sources 202 do not block light radiating through the tissue of the patient to be received by the photodiodes 206. For example, the light sources 202 and photodiodes 206 can be disposed in an alternating pattern around the syringe 300. The photodiodes 206 are electrically coupled to and in communication with a controller 320 of the syringe 300 to provide data associated with the light received when the drug delivery member is in the injection position thereto. The controller 320, or optionally a remote controller, can then correlate the data to a depth that the needle 312 has penetrated the skin 320 of the patient. It will be understood that the above sensing assembly 200 can be incorporated into an autoinjector device 10 with the array of light sources 202 and photodiodes 206 positioned relative to the primary container 12 and needle 20.

With the above forms, the controller 28, 70, 320 can determine, with reference to expected values, a depth of the drug delivery member 20, 62, 312 based on the provided measurements to thereby confirm and record a depth of the drug delivery member 20, 62, 312 during a drug dispensing operation. In some instances, delivery depth of a drug, such as to intradermal, subcutaneous, intramuscular, or intravenous sites, for example, can influence pharmacokinetics, which can result in therapeutic differences for a particular drug. Utilizing devices as described herein, interested parties can confirm, with data, that the device 10, 50, 300 was positioned correctly and that the drug delivery member 20, 62, 312 was inserted to a target depth.

The term controller refers broadly to any microcontroller, computer, or processor-based device with processor, memory, and programmable input/output peripherals, which is generally designed to govern the operation of other components and devices. It is further understood to include common accompanying accessory devices, including a power source, memory, transceivers for communication with other components and devices, etc. These architectural options are well known and understood in the art and require no further description here. The controllers may be configured (for example, by using corresponding programming stored in a memory as will be well understood by those skilled in the art) to carry out one or more of the steps, actions, and/or functions described herein.

It will be appreciated that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. The same reference numbers may be used to describe like or similar parts. Further, while several examples have been disclosed herein, any features from any examples may be combined with or replaced by other features from other examples. Moreover, while several examples have been disclosed herein, changes may be made to the disclosed examples within departing from the scope of the claims.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Solids™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF α monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3× epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive con

1. A drug delivery device comprising:
a housing;
a primary container disposed within the housing;
a drug delivery member fluidly coupled to the primary container, the drug delivery member movable between a retracted position disposed in the housing and an injection position at least partly extending out of the housing;
a wire having a first end and a second end, the second end electrically connected at a connection point adjacent to the drug delivery member, the connection point fixed against movement relative to the housing; and
a controller in communication with the first end of the wire, the controller configured to receive from the wire capacitance information associated with the drug delivery member in the injection position.

2. The drug delivery device of claim 1, wherein the controller is further configured to correlate the capacitance information to a depth that the drug delivery member has been inserted into a patient.

3. The drug delivery device of claim 1, further comprising a needle insertion mechanism; and
wherein the drug delivery member has an elongate configuration with a proximal end extending from the primary container and being fixed against movement with respect to the housing, an intermediate bend, and a distal end, the needle insertion mechanism configured to move at least a portion of the distal end between the retracted position and the injection position; and wherein the second end of the wire is fixed to the connection point at the proximal end of the drug delivery member.

4. The drug delivery device of claim 1, further comprising a pair of capacitor plates disposed within the housing and spaced from the drug delivery member, the drug delivery member extending between the pair of capacitor plates; and
wherein the connection point is located on the pair of capacitor plates, and the controller is in communication with the capacitor plates to receive capacitance information associated with the drug delivery member in the injection position.

5. The drug delivery device of claim 4, further comprising one or more dielectric members disposed between the capacitor plates and the drug delivery member, the one or more dielectric members spaced outwardly from the drug delivery member.

6. The drug delivery device of claim 1, wherein the drug delivery member comprises a cannula having a conductive portion and the connection point is adjacent to the conductive portion of the cannula.

7. The drug delivery device of claim 6, wherein the conductive portion of the cannula comprises a conductive coating extending over at least a portion of an outer surface of the cannula.

8-12. (canceled)

13. A drug delivery device comprising:
a housing;
a hub movably disposed within the housing;
a drug delivery member having a portion extending through and connected to the hub;
a needle insertion mechanism operably coupled to the hub and configured to move the hub to drive the drug delivery member between a retracted position disposed in the housing and an injection position at least partly extending out of the housing;
a wire with a first end and a second end, at least a portion of the second end of the wire fixed to the hub and electrically connected to the portion of the drug delivery member extending through the hub; and
a controller in communication with the wire to receive capacitance information associated with the drug delivery member in the injection position.

14. The drug delivery device of claim 13, wherein the controller is configured to correlate the capacitance information to a depth that the drug delivery member has been inserted into a patient.

15. The drug delivery device of claim 13, wherein the drug delivery member comprises a cannula having a conductive portion and the wire is electrically connected to the conductive portion of the cannula.

16. The drug delivery device of claim 15, wherein the conductive portion of the cannula comprises a conductive coating extending over at least a portion of an outer surface of the cannula.

17-19. (canceled)

20. A drug delivery device comprising:
a hub;
a drug delivery member fixed to the hub and having a proximal opening and a distal opening, the proximal opening disposed within the hub and in communication with the distal opening;
a light source oriented to project light into the proximal opening of the drug delivery member, out of the distal opening of the drug delivery member, and into the tissue of a patient with the drug delivery member in an injection position;
a photodiode oriented to receive light radiating through the tissue of the patient adjacent to the drug delivery member when the drug delivery member is in the injection position; and
a controller in communication with the photodiode to receive data associated with the light received.

21. The drug delivery device of claim 20, wherein the controller is configured to correlate the data to a depth that the drug delivery member has been inserted into the tissue of the patient.

22. The drug delivery device of claim 20, further comprising:
a housing;
a primary container disposed within the housing; and
a flowpath fluidly coupling the primary container to the drug delivery member;
wherein the drug delivery member extends through the hub, the drug delivery member including a bend disposed within the hub with the proximal opening extending therethrough and a distal end extending from the bend through a bottom surface of the hub.

23. The drug delivery device of claim 20, further comprising:
a housing;
a primary container disposed within the housing;
a flowpath fluidly coupled to the primary container; and
an inlet conduit mounted to hub;
wherein the hub includes an internal cavity, the inlet conduit fluidly couples the flowpath to the internal cavity of the hub, and the drug delivery member extends from the internal cavity through a bottom surface of the hub, the proximal opening of the drug delivery member fluidly coupling the drug delivery member to the internal cavity.

24. The drug delivery device of claim 22, wherein the photodiode is mounted to an upper surface of the hub.

25. The drug delivery device of claim 22, wherein the photodiodes is mounted to a bottom wall of the housing adjacent to a drug delivery member opening extending therethrough.

26. The drug delivery device of claim 20, further comprising a primary container, and wherein the drug delivery member comprises a needle, the hub is fixedly mounted to a distal end of the primary container, the light source comprises an array of light sources carried by the hub; and the photodiode comprises an array of photodiodes extending around and adjacent to the distal end of the primary container, the array of light sources and the array of photodiodes being non-aligned to provide generally unobstructed paths toward a distal end of the needle.

27-33. (canceled)
